(19) United States
(12) Patent Application Publication
(10) Pub. No.: US 2005/0255134 A1
(43) Pub. Date: Nov. 17, 2005

(54) Brushable cosmetic preparation having a high water content

(75) Inventors: Steffen Hasenzahl, Hanau (DE); Alfred Braunagel, Mainz (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/998,551

(22) Filed: Nov. 30, 2004

(30) Foreign Application Priority Data
Dec. 1, 2003 (DE) 103 56 647.3

(51) Int. Cl. A61K 7/00

(52) U.S. Cl. 424/401

(57) ABSTRACT

Brushable cosmetic preparation having a creamy or paste-like consistency, containing 50 to 95 wt. % of water, hydrophobed silicon dioxide powder, at least one cosmetically relevant active ingredient or auxiliary substance that is water-soluble or is dispersible or emulsifiable in an aqueous medium, and a viscosity regulator. It is produced by adding at least one cosmetically relevant active ingredient and/or auxiliary substance and optionally at least one pigment in water to a solution or dispersion adding hydrophobed silicon powder in portions or continuously whilst mixing and continuing mixing until the mixture, which is originally in powder form after addition of hydrophobed silicon dioxide powder, takes on a pasty consistency, and then adding a viscosity regulator and optionally at least one oil body, optionally before, together with or after addition of the viscosity regulator, with mixing. It can be used in pastes, creams, gels, pencils, foams, emulsions, dispersions, roll-ons, lotions.

The present invention concerns a brushable cosmetic preparation having a creamy consistency, its production and use.

### PRIOR ART

It is known that cosmetic preparations such as creams, lotions, pastes, mousses, etc., generally contain relatively large amounts of cosmetic oils and/or emulsifiers. Cosmetic products having a relatively high oil content are unsuitable for oily skin types in particular, or for skin that is prone to blackheads, spots or acne, for example. Furthermore, formulations containing emulsifiers can lead to allergic reaction.

The production of solid cosmetic preparations, such as powders, having a high water content is also known. The basis of some patent applications is the long-known fact that in the presence of hydrophobic silicon dioxide powder, water is dispersed into fine droplets and enveloped, and a joining together of the water droplets is thus prevented. A powdery substance, known as “dry water”, is formed in this way (Fine Particles series no. 11, Degussa AG, Dusseldorf).

Cosmetic formulations based on “dry water” are described in Seifen, Fette, Ole, Wachse (SOFW), 8 (2003), pages 1-8.

These are free-flowing, fine powders, which liquefy when rubbed on the skin. (Interestingly we have not actually said anything about the consistency of our product—it is creamy—so we can leave it like that.)

Water-containing, liquefiable powder compositions based on this principle are also described in EP-A-1235554, EP-A-1206928, JP 09-221404.

All these products are powders. The disadvantage of this application form is that it is difficult to apply the product completely and uniformly onto the skin, especially in the case of products for the face.

The object of the invention is therefore to provide an application form that avoids the disadvantage of the prior art. A further object of the invention is to provide a process for this application form.

The invention provides a brushable cosmetic preparation having a creamy or paste-like consistency, containing
50-95 wt. % water,
hydrophobed silicon dioxide powder,
at least one cosmetically relevant active ingredient or auxiliary substance that is water-soluble or is dispersible or emulsifiable in an aqueous medium and
a viscosity regulator.

The water content can preferably be between 75 and 90 wt. %.

Hydrophobed Silicon Dioxide Powder

There is no restriction on the type of hydrophobed silicon dioxide powder, provided that when it is added to water a product is formed that is initially in powder form.

Preparations according to the invention cannot be produced without the presence of hydrophobed silicon dioxide powder. Instead, low-viscosity solutions or dispersions are formed, which are not sufficiently stable.

The hydrophobed silicon dioxide powders can preferably be silanised. Halosilanes, alkoxysilanes, silazanes and/or siloxanes can be used for silanisation.

In particular, the following substances can be used as halosilanes:
Organohalosilanes of the type X₃Si(CₙH₂ₙ₊₁) where X=Cl, Br and n=1-20,
Organohalosilanes of the type X₂R′)Si(CₙH₂ₙ₊₁) where X=Cl, Br and R′=alkyl, n=1-20
Organohalosilanes of the type X(R′)₂Si(CₙH₂ₙ₊₁) where X=Cl, Br, R′=alkyl, n=1-20
Organohalosilanes of the type X₃Si(CH₂)ₘ—R′ where X=Cl, Br, m=0.1-20, R′=alkyl, aryl (for example —C₆H₅), —C₄F₉, —OCF₂—CHF—CF₃, —C₆F₁₃, —O—CF₂—CHF₂, —NH₂, —N₃, —SCN, —CH═CH₂, —OC(CH₃)C═CH₂, —OCH₂—CH(O)CH₂, —NH—COO—CH₃, —NH—COO—CH₂—CH₃, —NH—(CH₂)₃Si(OR)₃, —Sₓ—(CH₂)₃Si(OR)₃, 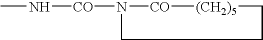
Organohalosilanes of the type (R)X₂Si(CH₂)ₘ—R′ where X=Cl, Br, R=alkyl, m=0.1-20, R′=alkyl, aryl (for example —C₆H₅), —C₄F₉, —OCF₂—CHF—CF₃, —C₆F₁₃, —O—CF₂—CHF₂, —NH₂, —N₃, —SCN, —CH═CH₂, —OC(CH₃)C═CH₂, —OCH₂—CH(O)CH₂, —NH—COO—CH₃, —NH—COO—CH₂—CH₃, —NH—(CH₂)₃Si (OR)₃, —NH—COO—CH₃, —NH—COO—CH₂—CH₃, —NH—(CH₂)₃Si(OR)₃, —Sₓ—(CH₂)₃Si(OR)₃ 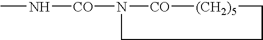
Organohalosilanes of the type (R)₂X Si(CH₂)ₘ—R′ where X=Cl, Br, R=alkyl, m=0.1-20, R′=alkyl, aryl (for example —C₆H₅), —C₄F₉, —OCF₂—CHF—CF₃, —C₆F₁₃, —O—CF₂—CHF₂, —NH₂, —N₃, —SCN, —CH═CH₂, —OC(CH₃)C═CH₂, —OCH₂—CH(O)CH₂, —NH—COO—CH₃, —NH—COO—CH₂—CH₃, —NH—(CH₂)₃Si(OR)₃, —Sₓ—(CH₂)₃Si(OR)₃,

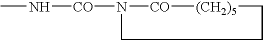

In particular, the following substances can be used as alkoxysilanes:
Organosilanes of the type (RO)₃Si(CₙH₂ₙ₊₁) where R=alkyl, n=1-20
Organosilanes of the type R′ₓ(RO)_{y}Si(CₙH₂ₙ₊₁) where R=alkyl, R′=alkyl, n=1-20, x+y=3, x=1, 2, y=1, 2
Organosilanes of the type (RO)₃Si(CH₂)ₘ—R′ where R=alkyl, m=0.1-20, R′=alkyl, aryl (for example —C₆H₅), —C₄F₉, OCF₂—CHF—CF₃, —C₆F₁₃, —O—CF₂—CHF₂, —NH₂, —N₃, —SCN, —CH═CH₂, —OC(CH₃)C═CH₂, —OCH₂—CH(O)CH₂, —NH—COO—CH₃, —NH—COO—CH₂—CH₃, —NH—(CH₂)₃Si(OR)₃, —Sₓ—(CH₂)₃Si(OR)₃ 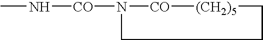
Organosilanes of the type (R″)ₓ(RO)_{y}Si(CH₂)ₘ—R′ where R″=alkyl, x+y=2, x=1, 2, y=1, 2, R′=alkyl, aryl (for example —C₆H₅), —C₄F₉, —OCF₂—CHF—CF₃, —C₆F₁₃, —O—CF₂—CHF₂, —NH₂, —N₃, —SCN, —CH═CH₂, —OC(CH₃)C═CH₂, —OCH₂CH(O)CH₂, NH—COO—CH₃, —NH—COO—CH₂—CH₃, —NH—(CH₂)₃Si(OR)₃, —Sₓ—(CH₂)₃Si(OR)₃

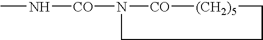

Trimethoxyoctyl silane [(CH₃O)₃—S₁—C₈H₁₇] (e.g. DYNASYLAN® OCTMO, Degussa AG) can preferably be used as silanising agent.

In particular, the following substances can be used as silazanes:

Silazanes of the type: 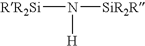
where R=alkyl, R′=alkyl, vinyl, and e.g. hexamethyl disilazane (for example DYNASYLAN® HMDS).

In particular, the following substances can be used as siloxanes:

Cyclic polysiloxanes of the type D 3, D 4, D 5, for example octamethyl cyclotetrasiloxane=D 4

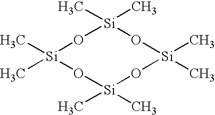

Polysiloxanes or silicone oils of the type: 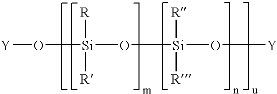
R=alkyl, aryl, (CH₂)ₙ—NH₂, H
R′=alkyl, aryl, (CH₂)ₙ—NH₂, H
R″=alkyl, aryl, (CH₂)ₙ—NH₂, H
R′″=alkyl, aryl, (CH₂)ₙ—NH₂, H
Y═CH₃, H, CₙH₂ₙ₊₁ where n=1-20
Y=Si(CH₃)₃, Si(CH₃)₂H
   Si(CH₃)₂OH, Si(CH₃)₂(OCH₃)
   Si(CH₃)₂(CₙH₂ₙ₊₁) where n=1-20
m=0, 1, 2, 3, . . . ∞
n=0, 1, 2, 3, . . . ∞
u=0, 1, 2, 3, . . . ∞

Silanisation can be performed by spraying the silicon dioxide with the silanising agent, which can optionally be dissolved in an organic solvent, such as ethanol for example, and then heat treating the mixture at a temperature of 105 to 400° C. for a period of 1 to 6 h.

The hydrophobed silicon dioxide powders preferably display a methanol wettability of at least 40. To determine the methanol wettability, 0.2 g (±0.005 g) of hydrophobic silicon dioxide power are weighed into individual transparent centrifuge tubes. 8.0 ml of a methanol/water mixture containing 10, 20, 30, 40, 50, 60, 70 and 80 vol. % methanol respectively are added to each weighed portion. The closed tubes are shaken for 30 seconds and then centrifuged for 5 minutes at 2500 rpm. The sediment volumes are read off, converted to a percentage and plotted against the methanol content (vol. %). The turning point of the curve corresponds to the methanol wettability.

Silicon dioxide powders hydrophobed with octamethyl cyclotetrasiloxane, polydimethyl siloxane, octyl silane and/or hexamethyl disilazane can particularly preferably be used.

There is no restriction on the specific surface area of the hydrophobed silicon dioxide powders. The preparation according to the invention preferably contains such powders having a specific surface area of between 10 and 400 and particularly preferably of 80 to 300 m²/g.

Examples of such hydrophobed silicon dioxide powders are Aerosil® R106, Aerosil® R202, Aerosil® R805, Aerosil® R812, Aerosil® R812S, Aerosil® R8200.

The preparation according to the invention can preferably display hydrophobed silicon dioxide powders having a compacted bulk density of at least 70 g/l, particularly preferably of 70 g/l to 250 g/l and most particularly preferably of 90 g/l to 180 g/l. Compacted bulk densities in the order of at least 70 g/l can be obtained for example by a mechanical aftertreatment of the silicon dioxide powder before or after hydrophobing. This can be a compaction, a structural modification or a granulation. According to the invention the compacted bulk density is determined in accordance with DIN EN ISO 787-11.

Preferred compacting processes are described for example in DE-A-3238427 and DE-A-3741846. Hydrophobed silicon dioxide powders compacted by means of the vacuum rotary filter described in DE-A-3741846, which is fitted with a pressing belt, are particularly advantageous.

Suitable commercially available, hydrophobed silicon dioxide powders can be Aerosil® R104 V (90), Aerosil® R202 VV90 (90), Aerosil® R805 VV90 (90), Aerosil R 812 S VV 75 (75), Aerosil® R812 VV90 (90), Aerosil® R812S VV90 (90), Aerosil® R8200 (140), all Degussa, or HDK® H2000 (approx. 200), HDK® H2050 (approx. 200), HDK® H3004 (approx. 100), all Wacker. Compacted bulk density in g/l in brackets.

It has been found that preparations according to the invention which display hydrophobed silicon dioxide powders having a compacted bulk density of at least 70 g/l, have a particularly long shelf life.

The proportion of hydrophobed silicon dioxide powder in the preparation according to the invention is preferably 1 to 15 wt. %.

A further constituent in the preparation according to the invention is at least one cosmetically relevant active ingredient or auxiliary substance that is water-soluble or is dispersible in an aqueous medium. The cosmetically relevant active ingredients and auxiliary substances are chosen from the group comprising
1. UV light stabilising filters,
2. Dyes and pigments,
3. Humectants/skin moisturising agents,
4. Deodorising and antiperspirant agents,
5. Biogenic substances,
6. Insect repellent agents,
7. Hydrotropes,
8. Anti-dandruff agents,
9. Bleaching or skin lightening agents and self-tanning agents,
10. Preservatives,
11. Surfactants/emulsifiers and/or
12. Perfume oils and plant extracts.

The proportion of cosmetically relevant active ingredient or auxiliary substance that is water-soluble, water-miscible or dispersible or emulsifiable in an aqueous medium in the preparation according to the invention can be 1 to 25 wt. %.

1. UV Light Stabilising Filters

UV light stabilising filters according to the invention are organic substances (light stabilising filters) which are liquid or crystalline at room temperature and which are capable of absorbing ultraviolet rays and of giving off the absorbed energy again in the form of longer-wave radiation, for example heat. UV filters can be oil-soluble or water-soluble. Examples of oil-soluble substances that can be cited are:
3-benzylidene camphor or 3-benzylidene norcamphor and derivatives thereof, for example 3-(4-methyl benzylidene) camphor as described in EP 0 693 471 B1
4-aminobenzoic acid derivatives, preferably 4-(dimethylamino)benzoic acid-2-ethylhexyl ester, 4-(dimethylamino)benzoic acid-2-octyl ester and 4-(dimethylamino)benzoic acid amyl ester
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene)
esters of salicylic acid, preferably salicylic acid-2-ethylhexyl ester, salicylic acid-4-isopropyl benzyl ester, salicylic acid homomenthyl ester
derivatives of benzophenone, preferably 2-hydroxy-4-methoxybenzophenone, 2-hydroxy-4-methoxy-4′-methyl benzophenone, 2,2′-dihydroxy-4-methoxybenzophenone
esters of benzalmalonic acid, preferably 4-methoxybenzalmalonic acid di-2-ethylhexyl ester
triazine derivatives, such as e.g. 2,4,6-trianilino-(p-carbo-2′-ethyl-1′-hexyloxy)-1,3,5-triazine and octyl triazone, as described in EP 0 818 450 A1 or dioctyl butamido triazone (Uvasorb™ HEB)
propane-1,3-diones, such as e.g. 1-(4-tert-butylphenyl)-3-(4′-methoxyphenyl)propane-1,3-dione
ketotricyclo(5.2.1.0)decane derivatives, as described in EP 0 694 521 B1.

Suitable water-soluble substances include:
2-phenyl benzimidazole-5-sulfonic acid and alkali, alkaline-earth, ammonium, alkyl ammonium, alkanol ammonium and glucammonium salts thereof
sulfonic acid derivatives of benzophenones, preferably 2-hydroxy-4-methoxybenzophenone-5-sulfonic acid and salts thereof
sulfonic acid derivatives of 3-benzylidene camphor, such as e.g. 4-(2-oxo-3-bornylidene methyl) benzenesulfonic acid and 2-methyl-5-(2-oxo-3-bornylidene)sulfonic acid and salts thereof.

Typical examples of UV-A filters are in particular derivatives of benzoyl methane, such as e.g. 1-(4′-tert-butylphenyl)-3-(4′-methoxyphenyl)propane-1,3-dione, 4-tert-butyl-4′-methoxydibenzoyl methane (Parsol™ 1789), 1-phenyl-3-(4′-isopropylphenyl)-propane-1,3-dione and enamine compounds, as described in DE 191 12 033 A1 (BASF). The UV-A and UV-B filters can naturally also be used in mixtures. Particularly favourable combinations consist of derivatives of benzoyl methane, for example 4-tert-butyl-4′-methoxydibenzoyl methane (Parsol™ 1789) and 2-cyano-3,3-phenyl cinnamic acid-2-ethylhexyl ester (Octocrylene) in combination with esters of cinnamic acid, preferably 4-methoxycinnamic acid-2-ethylhexyl ester and/or 4-methoxycinnamic acid propyl ester and/or 4-methoxycinnamic acid isoamyl ester. Such combinations are advantageously combined with water-soluble filters, such as e.g. 2-phenyl benzimidazole-5-sulfonic acid and alkali, alkaline-earth, ammonium, alkyl ammonium, alkanol ammonium and glucammonium salts thereof.

Particularly advantageous according to the invention are UV filters which can be dissolved or emulsified in the aqueous phase.

In addition to the cited soluble substances, insoluble light stabilising pigments, namely finely dispersed metal oxides or salts, are also suitable for this purpose.

Examples of suitable metal oxides are in particular zinc oxide and titanium dioxide and also oxides of iron, zirconium, silicon, manganese, aluminium and cerium, as well as mixtures thereof. Silicates (talc), barium sulfate or zinc stearate can be used as salts.

So-called micropigments or nanopigments are preferably used in sunscreens. The particles should display an average diameter of less than 100 nm, preferably between 5 and 50 nm and in particular between 15 and 30 nm. They can display a spherical form, but such particles having an ellipsoid form or other form deviating from the spherical shape can also be used.

The pigments can also be surface treated, with water-wettable pigments being particularly preferred. Typical examples are coated titanium dioxides such as e.g. UV-titanium M212, M 262 and X 111 (Kemira), AEROXIDE TiO2 P25, PF2, T 805 and T 817 (Degussa), micro titanium dioxide MT-150 W, MT-100 AQ, MT-100 SA, MT-100 HD, MT-100 TV (Tayca), Eusolex™ T2000 (Merck), zinc oxide neutral H&R and zinc oxide NDM (Haarmann & Reimer) and Z-Cote and Z-Cote HP1 (BASF). Dispersions such as TEGO Sun TAQ 40, for example, a 40 wt. % aqueous dispersion of a hydrophobed titanium dioxide (Degussa), can also be used. Other suitable UV light-stabilising filters can be found in the survey by P. Finkel in SOFW-Journal 122, 543 (1996) and Parf. Kosm. 3, 11 (1999). Optical brighteners such as e.g. 4,4′-diaminostilbene-2,2′-disulfonic acid and derivatives thereof can also be used.

In addition to the two groups of primary light stabilising substances cited above, secondary light stabilisers of the antioxidant type can also be used, which interrupt the photochemical reaction chain that is initiated when UV radiation penetrates the skin. Typical examples of these are amino acids (for example glycine, histidine, tyrosine, tryptophane) and derivatives thereof, imidazoles (for example urocanic acid) and derivatives thereof, peptides such as D,L-carnosine, D-carnosine, L-carnosine and derivatives thereof (for example anserine), carotenoids, carotenes (for example alpha-carotene, beta-carotene, lycopene) and derivatives thereof, chlorogenic acid and derivatives thereof, lipoic acid and derivatives thereof (for example dihydrolipoic acid), aurothioglucose, propyl thiouracil and other thiols (for example thioredoxine, glutathione, cysteine, cystine, cystamine and glycosyl, N-acetyl, methyl, ethyl, propyl, amyl, butyl and lauryl, palmitoyl, oleyl, gamma-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine sulfoximines, homocysteine sulfoximine, buthionine sulfones, penta-, hexa-, heptathionine sulfoximine) in very small tolerated doses (for example pmol to mu mol/kg), also (metal) chelators (for example alpha-hydroxy fatty acids, palmitic acid, phytic acid, lactoferrin), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example gamma-linolenic acid, linoleic acid, oleic acid), folic acid and derivatives thereof, ubiquinone and ubiquinol and derivatives thereof, vitamin C and derivatives (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoic resin, rutinic acid and derivatives thereof, alpha-glycosyl rutin, ferulic acid, furfurylidene glucitol, carnosine, butyl hydroxytoluene, butyl hydroxyanisole, nordihydroguaiac acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and the derivatives (salts, esters, ethers, sugars, nucleotides, nucleosides, peptides and lipids) of these cited active ingredients that are suitable according to the invention.

2. Dyes and Pigments

Dyes that can be used according to the invention are natural, vegetable or animal dyes such as, for example, betanin, bixin, carmine, carotene, chlorophyll, sepia, etc. and derivatives thereof, as well as synthetic organic dyes, such as e.g. azo, anthraquinone, triphenylmethane dyes, etc. Dyes that are water-soluble or are dispersible in water can be particularly preferred.

The preparation according to the invention can also contain inorganic pigments, such as ochre, umber, red bole, sienna, chalk, etc. and synthetic inorganic pigments such as iron oxides, ultramarines, titanium dioxide, zinc oxide, mica-based pigments, such as e.g. pearlescent pigments. Water-wettable pigments can be particularly preferred.

3. Humectants/Skin Moisturising Agents

In a preferred embodiment the preparation according to the invention also contains a humectant. This serves to further optimise the sensory properties of the composition and to regulate the moisture of the skin. At the same time the low-temperature stability of the preparations according to the invention, especially in the case of emulsions, is increased. The humectants are conventionally included in an amount of 0.1 to 15 wt. %, preferably 1 to 10 wt. %, and in particular 5 to 10 wt. %.

Suitable according to the invention are, inter alia, amino acids, pyrrolidone carboxylic acid, lactic acid and salts thereof, lactitol, urea and urea derivatives, uric acid, glucosamine, creatinine, breakdown products of collagen, chitosan or chitosan salts/derivatives, and in particular polyols and polyol derivatives (for example glycerol, diglycerol, triglycerol, ethylene glycol, propylene glycol, butylene glycol, erythritol, 1,2,6-hexanetriol, polyethylene glycols such as PEG-4, PEG-6, PEG-7, PEG-8, PEG-9, PEG-10, PEG-12, PEG-14, PEG-16, PEG-18, PEG-20), sugars and sugar derivatives (inter alia fructose, glucose, maltose, maltitol, mannitol, inositol, sorbitol, sorbityl silanediol, sucrose, trehalose, xylose, xylitol, glucuronic acid and salts thereof), ethoxylated sorbitol (Sorbeth-6, Sorbeth-20, Sorbeth-30, Sorbeth-40), honey and hydrogenated honey, hydrogenated starch hydrolysates and mixtures of hydrogenated wheat protein and PEG-20 acetate copolymer.

The preparation according to the invention can particularly preferably contain glycerol, diglycerol, triglycerol and butylene glycol.

4. Deodorising and Antiperspirant Agents

Deodorising and antiperspirant agents can also be added according to the invention. These active ingredients include astringent metal salts (antiperspirant agents), microbe-inhibiting agents, enzyme inhibitors, odour absorbers, odour maskers or any combination of these active ingredients. The deodorising/antiperspirant agents can be included in the preparation according to the invention in an amount from 0.1 to 30 wt. %, preferably 5 to 25 wt. % and in particular 10 to 25 wt. % (relative to the amount of preparation).

Aluminium chlorohydrates, aluminium zirconium chlorohydrates and zinc salts, for example, can be used as antiperspirant agents. In addition to the chlorohydrates, the preparation according to the invention can also contain aluminium hydroxylactates and acid aluminium/zirconium salts, for example Locron™ (formula [Al₂(OH)₅Cl]x2.5 H₂O, Clariant GmbH) or Rezal™ 36G (aluminium zirconium tetrachlorohydrex glycine complexes, Reheis).

Enzyme inhibitors, for example esterase inhibitors, can be added as additional deodorising agents. These are preferably trialkyl citrates, such as trimethyl citrate, tripropyl citrate, triisopropyl citrate, tributyl citrate and in particular triethyl citrate (Hydagen™ C. A. T., Cognis Deutschland GmbH). The substances inhibit the enzyme activity of sweat-decomposing bacteria, thereby reducing the formation of odours. Other substances that can be considered as esterase inhibitors are sterol sulfates or phosphates, such as e.g. lanosterol, cholesterol, campesterol, stigmasterol and sitosterol sulfate or phosphate, dicarboxylic acids and esters thereof, such as e.g. glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, such as e.g. citric acid, malic acid, tartaric acid or tartaric acid diethyl ester. Antibacterial agents, which influence microbial flora and kill or inhibit the growth of sweat-decomposing bacteria, can likewise be included in the preparation according to the invention. Examples of these are chitosan, phenoxyethanol, chlorohexidine gluconate or 5-chloro-2-(2,4-dichlorophenoxy) phenol (Irgasan™, Ciba-Geigy, Basle/CH).

All substances that are active against gram-positive bacteria are suitable in principle as microbe-inhibiting agents, such as e.g. 4-hydroxybenzoic acid and its salts and esters, N-(4-chlorophenyl)-N′-(3,4 dichlorophenyl) urea, 2,4,4′-trichloro-2′-hydroxydiphenyl ether (triclosan), 4-chloro-3,5-dimethylphenol, 2,2′-methylene bis-(6-bromo-4-chlorophenol), 3-methyl-4-(1-methylethyl) phenol, 2-benzyl-4-chlorophenol, 3-(4-chlorophenoxy)-1,2-propanediol, 3-iodine-2-propynyl butyl carbamate, chlorohexidine, 3,4,4′-trichlorocarbanilide (TTC), antibacterial perfumes, thymol, thyme oil, eugenol, clove oil, menthol, mint oil, farnesol, phenoxyethanol, glycerol monocaprinate, glycerol monocaprylate, glycerol monolaurate (GML), diglycerol monocaprinate (DMC), salicylic acid-N-alkylamides such as e.g. salicylic acid-n-octylamide or salicylic acid-n-decylamide.

Substances that can absorb and largely retain odour-producing compounds are suitable as odour absorbers. They lower the partial pressure of the individual components, thereby also reducing their speed of propagation. It is important here that perfumes are not adversely affected. As their main component, for example, they contain a complex zinc salt of ricinoleic acid or special, largely odour-neutral aromatics, which are known to the person skilled in the art as “fixatives”, such as e.g. extracts of ladanum or styrax or certain abietic acid derivatives.

Fragrances or perfume oils act as odour maskers which, in addition to their function as odour maskers, give the deodorants their scent. Examples of perfume oils that can be cited by way of example are mixtures of natural and synthetic fragrances. Natural fragrances are extracts of flowers, stems and leaves, fruits, fruit skins, roots, woods, herbs and grasses, needles and twigs as well as resins and balsams. Animal raw materials are also suitable, such as e.g. civet and castoreum. Typical synthetic fragrance compounds are products of the ester, ether, aldehyde, ketone, alcohol and hydrocarbon type. Fragrance compounds of the ester type are for example benzyl acetate, p-tert-butyl cyclohexyl acetate, linalyl acetate, phenyl ethyl acetate, linalyl benzoate, benzyl formate, allyl cyclohexyl propionate, styrallyl propionate and benzyl salicylate. The ethers include, for example, benzyl ethyl ethers, the aldehydes include, for example, the linear alkanals having 8 to 18 carbon atoms, citral, citronellal, citronellyl oxyacetaldehyde, cyclamen aldehyde, hydroxy citronellal, lilial and bourgeonal, the ketones include the ionones and methyl cedryl ketone, for example, the alcohols include anethol, citronellol, eugenol, isoeugenol, geraniol, linalool, phenylethyl alcohol and terpineol, the hydrocarbons primarily include the terpenes and balsams. Mixtures of various fragrances are preferably used, however, which together produce a pleasant scent. Ethereal oils having a relatively low volatility, which are mainly used as aroma components, are also suitable as perfume oils, for example sage oil, camomile oil, clove oil, melissa oil, mint oil, cinnamon leaf oil, lime blossom oil, juniper berry oil, vetiver oil, olibanum oil, galbanum oil, ladanum oil and lavendin oil. Bergamot oil, dihydromyrcenol, lilial, lyral, citronellol, phenyl ethyl alcohol, alpha-hexylcinnamaldehyde, geraniol, benzyl acetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, beta-damascone, geranium oil bourbon, cyclohexyl salicylate, Vertofix Coeur, Iso-E-Super, Fixolide NP, evernyl, iraldein gamma, phenyl acetic acid, geranyl acetate, benzyl acetate, rose oxide, romilat, irotyl and floramat are preferably used, alone or in mixtures.

5. Biogenic Substances

Biogenic active ingredients that are suitable according to the invention are for example tocopherol, tocopherol acetate, tocopherol palmitate, ascorbic acid, (deoxy)ribonucleic acid and fragmentation products thereof, beta-glucans, retinol, bisabolol, allantoin, phytanetriol, panthenol, panthotenic acid, alpha-hydroxy acid fruit acids, amino acids, ceramides, pseudoceramides, essential oils, plant extracts, such as e.g. prunus extract, bambara nut extract, and vitamin complexes.

6. Insect Repellent Agents

A further preferred embodiment of the preparation according to the invention additionally contains at least one insect repellent agent or a combination of these agents. Suitable insect repellents include, for example, N,N-diethyl-m-toluamide, 1,2-pentanediol or 3-(N-n-butyl-N-acetyl amino) propionic acid ethyl ester)(Insect Repellent 3535, Merck KGaA) and butyl acetyl aminopropionates. They are conventionally present in the preparation according to the invention in an amount of 0.1-10 wt. %, preferably 1-8 wt. % and particularly preferably in an amount of 2-6 wt. %, relative to the preparation.

7. Hydrotropes

Hydrotropes, such as ethanol, isopropyl alcohol or polyols for example, can also be included according to the invention. Polyols for consideration here preferably have 2 to 15 carbon atoms and at least two hydroxyl groups. The polyols can also contain other functional groups, in particular amino groups, or be modified with nitrogen. Typical examples are:
glycerol
alkylene glycols, such as e.g. ethylene glycol, diethylene glycol, propylene glycol, butylene glycol, hexylene glycol and polyethylene glycols having an average molecular weight of 100 to 1000 daltons
technical oligoglycerol mixtures having an intrinsic degree of concentration of 1.5 to 10, such as e.g. technical diglycerol mixtures having a diglycerol content of 40 to 50 wt. %
methylol compounds, such as in particular trimethylol ethane, trimethylol propane, trimethylol butane, pentaerythritol and dipentaerythritol
short-chain alkyl glucosides, in particular those having 1 to 8 carbons in the alkyl radical, such as e.g. methyl and butyl glucoside
sugar alcohols having 5 to 12 carbon atoms, such as sorbitol or mannitol, for example
sugars having 5 to 12 carbon atoms, such as glucose or sucrose, for example
amino sugars, such as glucamine for example
dialcohol amines, such as diethanolamine or 2-amino-1,3-propanediol.

8. Anti-Dandruff Agents

Suitable anti-dandruff agents in the preparation according to the invention include piroctone olamine (1-hydroxy-4-methyl-6-(2,4,4-trimethylpentyl)-2-(1H)-pyridinone monoethanolamine salt), Baypival™ (climbazole), Ketoconazol™, (4-acetyl-1-4-[2-(2.4-dichlorophenyl)-r-2-(1H-imidazol-1-yl methyl)-1,3-dioxylan-c-4-ylmethoxyphenyl piperazine, ketoconazole, elubiol, selenium disulfide, colloidal sulfur, sulfur polyethylene glycol sorbitane monooleate, sulfur ricinol polyethoxylate, sulfur tar distillate, salicylic acid (or in combination with hexachlorophene), undecylenic acid monoethanolamide sulfosuccinate Na salt, Lamepon™ UD (protein undecylenic acid condensate), zinc pyrithione, aluminium pyrithione and magnesium pyrithione/dipyrithione magnesium sulfate.

9. Bleaching or Skin Lightening Agents and Self-Tanning Agents

The preparation according to the invention can contain bleaching or skin lightening agents, such as e.g. basic bismuth salts, hydroquinone, oxygen-eliminating compounds, such as e.g. zinc peroxide, urea peroxide, hydrogen peroxide and/or organic peroxides. The preparation according to the invention can particularly preferably contain hydrogen peroxide, which is used in the form of aqueous solutions. Suitable examples of tyrosinase inhibitors, which prevent the formation of melanin and are used in depigmenting agents, include arbutin, ferulic acid, kojic acid, coumarinic acid and ascorbic acid (vitamin C, sodium ascorbyl phosphate, magnesium ascorbyl phosphate). Particularly suitable is Cosmocair C 250 from Degussa AG. Dihydroxyacetone, for example, is suitable as a self-tanning agent.

10. Preservatives

Suitable preservatives are, for example, phenoxyethanol, formaldehyde solution, parabens, pentanediol or sorbic acid and the silver complexes known under the name Surfacine™ and the other classes of substances listed in Annex 6, Part A and B of the German cosmetics ordinance.

11. Surfactants/Emulsifiers

The preparation according to the invention can contain surfactants/emulsifiers. The amount of these substances in the preparation is critical, however, since their wetting behaviour can prevent the formation of a powder when hydrophobed silicon dioxide powder is added, so that a preparation according to the invention cannot be obtained. The preparations according to the invention therefore generally contain no surfactants/emulsifiers. If they are to be included in the preparation according to the invention, the amount must be optimised according to the preparation.

There is no restriction on the type of surfactant/emulsifier. A preparation according to the invention can thus contain non-ionic, zwitterionic, amphoteric, cationic and also anionic surfactants.

12. Perfume Oils and Plant Extracts

The preparation according to the invention can contain perfume oils. These can be natural, plant and animal as well as synthetic fragrances or mixtures thereof. Natural fragrances are obtained inter alia by extraction from flowers, stems, leaves, fruit, fruit skins, roots and resins of plants. Animal raw materials are also suitable, such as e.g. civet and castoreum. Typical synthetic fragrance compounds are products of the ester, ether, aldehyde, ketone, alcohol and hydrocarbon type. Mixtures of various fragrances are preferably used, which together produce a pleasant scent.

Plant extracts that can be used according to the invention include, for example, extracts of arnica, birch, camomile, burr root, beard lichen, poplar, stinging nettle and walnut shells.

Viscosity Regulators

A substantial constituent of the preparation according to the invention are viscosity regulators. The preparation preferably contains hydrogel formers or hydrocolloids, such as e.g. modified polysaccharides such as cellulose ethers and cellulose esters, for example carboxymethyl cellulose, methyl cellulose, hydroxyethyl cellulose, methylhydroxyethyl cellulose, methylhydroxypropyl cellulose, xanthan gum, guar guar, agar agar, alginates and tyloses; inorganic hydrocolloids such as bentonites, magnesium aluminium silicates, silicon dioxide; and synthetic hydrocolloids such as polyacrylates (for example Carbopole™ and Pemulen types from Goodrich; Synthalene™ from Sigma; Keltrol types from Kelco; Sepigel types from Seppic; Salcare types from Allied Colloids), uncrosslinked and polyol-crosslinked polyacrylic acids, polyacrylamides, polyvinyl alcohol and polyvinyl pyrrolidone.

Surfactants such as e.g. ethoxylated fatty acid glycerides, esters of fatty acids with polyols, such as e.g. pentaerythritol or trimethylol propane, fatty alcohol ethoxylates with concentrated homologue distribution, alkyl oligoglucosides and electrolytes, such as e.g. common salt and ammonium chloride, can also be used for viscosity regulation.

Also suitable as viscosity regulators are anionic, zwitterionic, amphoteric and nonionic copolymers, such as e.g. vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and esters thereof, acrylamidopropyl trimethyl ammonium chloride/acrylate copolymers, octyl acrylamide/methyl methacrylate/tert-butyl aminoethyl methacrylate/2-hydroxypropyl methacrylate copolymers, vinyl pyrrolidone/vinyl acetate copolymers, vinyl pyrrolidone/dimethyl aminoethyl methacrylate/vinyl caprolactam terpolymers and optionally derivatised cellulose ethers and silicones. Other suitable polymers and thickeners are listed in Cosm. Toil. 108, 95 (1993).

The proportion of viscosity regulator in the preparation according to the invention can preferably be 1 to 5 wt. %.

Oil Bodies

The preparation according to the invention can also contain at least one oil body. According to the invention oil bodies are understood to be substances or mixtures of substances that are liquid at 20° C. and are immiscible with water at 25° C. The combination with oil bodies allows the sensory properties of the preparations to be optimised.

Examples of oil bodies include guerbet alcohols based on fatty alcohols having 6 to 18, preferably 8 to 10 carbon atoms (for example Eutanol™ G), esters of linear C₆-C₂₂ fatty acids with linear or branched C₆-C₂₂ fatty alcohols or esters of branched C₆-C₁₃ carboxylic acids with linear or branched C₆-C₂₂ fatty alcohols, such as e.g. myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂ fatty acids with branched alcohols, in particular 2-ethyl hexanol, esters of C₃-C₃₈ alkyl hydroxycarboxylic acids with linear or branched C₆-C₂₂ fatty alcohols—in particular diethylhexyl malate—, esters of linear and/or branched fatty acids with polyhydric alcohols (such as e.g. propylene glycol, dimerdiol or trimertriol) and/or guerbet alcohols, triglycerides based on C₆-C₁₀ fatty acids, liquid mono-/di-/triglyceride mixtures based on C₆-C₁₈ fatty acids, esters of C₆-C₂₂ fatty alcohols and/or guerbet alcohols with aromatic carboxylic acids, in particular benzoic acid, esters of C₂-C₁₂ dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂ fatty alcohol carbonates, such as e.g. dicaprylyl carbonates (Cetiol™ CC), guerbet carbonates based on fatty alcohols having 6 to 18, preferably 8 to 10 C atoms, esters of benzoic acid with linear and/or branched C₆-C₂₂ alcohols (for example Finsolv™ TN), linear or branched, symmetrical or asymmetrical dialkyl ethers having 6 to 22 carbon atoms per alkyl group, such as e.g. dicaprylyl ethers (Cetiol™ OE), ring-opening products of epoxidised fatty acid esters with polyols (Hydagen™ HSP, Sovermol™ 750, Sovermol™ 1102), and/or aliphatic or naphthenic hydrocarbons, such as e.g. mineral oil, vaseline, petrolatum, squalane, squalene, dialkyl ether dialkyl carbonates and/or dialkyl cyclohexanes.

The preparation according to the invention can moreover contain silicone compounds. These can be cyclomethicones, dimethicones, dimethyl polysiloxanes, methyl phenyl polysiloxanes, cyclic silicones, amino-, fatty acid-, alcohol-, polyether-, epoxy-, fluorine-, glycoside- and/or alkyl-modified silicone compounds. Also suitable are simethicones, which are mixtures of dimethicones having an average chain length of 200 to 300 dimethyl siloxane units and silicon dioxide or hydrogenated silicates.

Depending on the application form, the amount of oil bodies in the overall composition can be between 0.1 and 10 wt. %. The amount can particularly preferably vary between 0.5 and 3 wt. %.

The invention also provides a process for the production of the brushable, cosmetic preparation which is characterised in that
at least one cosmetically relevant active ingredient and/or auxiliary substance and optionally at least one pigment in water or in an aqueous solution are added to a solution, dispersion or emulsion
hydrophobed silicon powder is added in portions or continuously whilst mixing and
mixing is continued until the mixture, which is originally in powder form after addition of hydrophobed silicon dioxide powder, takes on a brushable creamy or paste-like consistency,
and then a viscosity regulator and optionally at least one oil body, optionally before, together with or after addition of the viscosity regulator, is added with mixing.

The substantial feature of the process is that mixing of the components is continued until the mixture, which is originally in powder form after addition of hydrophobed silicon dioxide powder, takes on a brushable creamy or pasty consistency.

The invention also provides the use of the brushable, cosmetic preparation in pastes, creams, gels, pencils, foams, emulsions, dispersions, roll-ons, lotions.

Suitable product forms can be:
skincare products
beauty masks
make-up foundations
bath and shower additives
hair removal products
antiperspirants, deodorants and perspiration-inhibiting agents,
hair treatment products, such as dyes and decolourising agents, hair bleaching products, perm lotions, smoothing products, setting lotions, cleaning products, styling products
shaving products, preshave and aftershave products,
make-up and make-up removal products for face and eyes, such as face make-up, tinted day cream, powder cream, cream make-up, blusher, eye shadows, mascara, kohl pencil, eyeliner or eyebrow pencil;
lipcare products and cosmetics, such as lipstick, lip gloss or lip liner;
dental and oral hygiene products
nailcare products and cosmetics, such as cuticle remover, nail hardener or nailcare cream
external intimate care products
sunscreens
sunless tanning products
anti-wrinkle products
insect repellents

### EXAMPLES

### Example 1

Water and Hydrolite-5 are Placed in a Mixer and the pigments added with stirring and then briefly dispersed with a homogeniser. AEROSIL® R 812 S is added with the homogeniser running and homogenised for a further 10 minutes until the initially powdered mixture has taken on a pasty consistency. ABIL® 20, ABIL® B 8843 and liquipar oil were added with stirring and the mixture was stirred for a further 2 to 3 minutes. Covagel was scattered in with stirring and the resulting mixture stirred for a further 15 minutes. A readily brushable, creamy and stable formulation is obtained in this process.

### Example 1a

AEROSIL® R 812 S replaced by AEROSIL® R 812 S VV60 with a compacted bulk density of 60 g/l.

### Example 1b

AEROSIL® R 812 S replaced by AEROSIL® R 812 S VV75 with a compacted bulk density of 75 g/l.

### Example 1c

AEROSIL® R 812 S replaced by AEROSIL® R 812 S VV90 with a compacted bulk density of 90 g/l.

### Example 1d

AEROSIL® R 812 S replaced by AEROSIL® R 8200 with a compacted bulk density of 140 g/l.

### Example 1e

AEROSIL® R 812 S replaced by a mixture of 8 parts by weight of AEROSIL® R 812 S VV90 with a compacted bulk density of 90 g/l and 2 parts by weight of AEROXIDE® TiO₂ T805.

### Example 1f

AEROSIL® R 812 S replaced by a mixture of 8 parts by weight of AEROSIL® R 812 S VV90 with a compacted bulk density of 90 g/l and 2 parts by weight of AEROXIDE® Alu C hydrophobed with hexamethyl disilazane and with a compacted bulk density of 80 g/l.

### Example 1g

AEROSIL® R 812 S replaced by a mixture of 8 parts by weight of AEROSIL® R 812 S VV90 with a compacted bulk density of 90 g/l and 2 parts by weight of AEROPERL R 806/30, a granulated, pyrogenic silicon dioxide hydrophobed with hexamethyl disilazane and with a compacted bulk density of approximately 300 g/l.

The preparations from examples 1, 1a-1g display good stability in storage. The preparations from examples 1b-1g, which contain hydrophobed silicon dioxide powders with a compacted bulk density of at least 70 g/l, display particularly good stability in storage. Furthermore, working with the hydrophobed silicon dioxide powders having a compacted bulk density of at least 70 g/l leads to only low dust formation, and the high flowability allows greater metering accuracy when producing the preparations.

### Example 2

Example 2 is performed in the same way as Example 1, with an amount of Abil 20 increased to 2.0 wt. %. As in Example 1, a readily brushable, creamy and stable formulation is obtained in this process.

### Comparative example C1

is performed in the same way as Example 2, except that after addition of the hydrophobed silicon dioxide the mixture is stirred for only five minutes. A powdered preparation is produced which liquefies when rubbed on the skin.

### Comparative example C2

is performed in the same way as Example 2, but without sodium carboxymethyl starch as viscosity regulator. A creamy formulation is produced, which is not stable in storage, however.

### Comparative example C3

is performed in the same way as Example 2, but without the addition of hydrophobed silicon dioxide. A low-viscosity suspension is produced, out of which the pigments settle very quickly. Hydrophobed silicon dioxide powder is therefore an essential constituent for producing a creamy and stable formulation.

### Example 3

is performed in the same way as Example 2, replacing dimethicone with ethylhexyl palmitate. As in Examples 1 and 2, a creamy, stable preparation is produced, which has a somewhat darker shade, however.

### Example 4

is performed in the same way as Example 2, but without the addition of dimethicone. As in Example 2, a creamy, stable formulation is produced.

### Examples 5 to 40

are produced in accordance with Examples 1 and 2. All formulations display a brushable consistency and are stable in storage.

### Examples 1, 1a-1g and 2

### Make-Up Foundation

| | | 1 | 2 |
|---|---|---|---|
| Raw material | INCI name | wt. % | wt. % |
| Demineralised water | Aqua (water) | 82.35 | 78.35 |
| Hydrolite-5 | Pentylene glycol | 5.00 | 5.00 |
| LCW AC-5 Yellow | CI 77492 & microcrystalline | 2.50 | 2.50 |
| LLXLO | cellulose & cellulose gum | | |
| LCW AC-5 Red | CI 77491 & microcrystalline | 0.75 | 0.75 |
| R-516 L | cellulose & cellulose gum | | |
| LCW AC-5 Black | CI 77499 & microcrystalline | 0.50 | 0.25 |
| BL-100 | cellulose & cellulose gum | | |
| LCW AC-5 TiO₂ | CI 77891 & aluminium | 2.00 | 2.25 |
| CR-50 | hydroxide & microcrystalline | | |
| | cellulose & cellulose gum | | |
| LCW AC-5 Sericite | Mica & microcrystalline | | 2.50 |
| FSE | cellulose & cellulose gum | | |
| Aerosil R 812 S | Silica silylate | 5.00 | 5.00 |
| Abil 20 | Dimethicone | 0.50 | 2.00 |
| Abil B 8843 | Dimethicone copolyol | 0.50 | 0.50 |
| Liquipar oil | Isobutylparaben & | 0.15 | 0.15 |
| | isopropylparaben & | | |
| | butylparaben | | |
| LCW Covagel | Sodium carboxymethyl starch | 0.75 | 0.75 |
| Total | | 100.00 | 100.00 |

### Comparative Examples 1 to 3, Examples 3 and 4

### According to the Invention

| | | C1 | C2 | C3 | 3 | 4 |
|---|---|---|---|---|---|---|
| Raw material | INCI name | wt. % | wt. % | wt. % | wt. % | wt. % |
| Demineralised water | Aqua (water) | 78.35 | 83.10 | 83.35 | 78.35 | 80.85 |
| Hydrolite-5 | Pentylene glycol | 5.00 | 5.00 | 5.00 | 5.00 | 5.00 |
| LCW AC-5 Yellow LLXLO | CI 77492 & microcrystalline | 2.50 | 2.50 | 2.50 | 2.50 | 2.50 |
| | cellulose & cellulose gum | | | | | |
| LCW AC-5 Red R-516 L | CI 77491 & microcrystalline | 0.75 | 0.75 | 0.75 | 0.75 | 0.75 |
| | cellulose & cellulose gum | | | | | |
| LCW AC-5 Black BL-100 | CI 77499 & microcrystalline | 0.50 | 0.50 | 0.50 | 0.50 | 0.50 |
| | cellulose & cellulose gum | | | | | |
| LCW Ac-5 TiO₂ CR-50 | CI 77891 & aluminium | 2.00 | 2.00 | 2.00 | 2.00 | 2.00 |
| | hydroxide & microcrystalline | | | | | |
| | cellulose & cellulose gum | | | | | |
| LCW AC-5 Sericite FSE | Mica & microcrystalline | 2.50 | | 2.50 | 2.50 | 2.50 |
| | cellulose & cellulose gum | | | | | |
| Aerosil R 812 S VV 60 | Silica silylate | 5.00 | 5.00 | 0.00 | 5.00 | 5.00 |
| Abil 20 | Dimethicone | 2.00 | 2.00 | 2.00 | 2.00 | 0.00 |
| Abil B 8843 | Dimethicone copolyol | 0.50 | 0.50 | 0.50 | 0.50 | 0.00 |
| Tegosoft OP | Ethylhexyl palmitate | | | | 2.00 | |
| Liquipar oil | “Parabens” | 0.15 | 0.15 | 0.15 | 0.15 | 0.15 |
| LCW Covagel | Sodium carboxymethyl starch | 0.75 | 0.00 | 0.75 | 0.75 | 0.75 |
| Total | | 100.0 | 100.0 | 100.0 | 100.0 | 100.00 |

### Examples 5-8

### Light Make-Up Foundation

| | | 5 | 6 | 7 | 8 |
|---|---|---|---|---|---|
| Raw material | INCI name | wt. % | wt. % | wt. % | wt. % |
| Demineralised water | Aqua (water) | 78.35 | 78.45 | 79.30 | 79.30 |
| Hydrolite-5 | Pentylene glycol | 5.00 | 5.00 | 5.00 | 5.00 |
| LCW AC-5 Yellow LLXLO | CI 77492 & microcrystalline | 1.25 | 1.25 | 0.60 | 0.40 |
| | cellulose & cellulose gum | | | | |
| LCW AC-5 Red R-516 L | CI 77491 & microcrystalline | 0.35 | 0.35 | 0.20 | 0.10 |
| | cellulose & cellulose gum | | | | |
| LCW AC-5 Black BL-100 | CI 77499 & microcrystalline | 0.10 | | | 0.03 |
| | cellulose & cellulose gum | | | | |
| LCW AC-5 TiO2 CR-50 | CI 77891 & aluminium | 4.05 | 4.05 | 4.00 | 4.27 |
| | hydroxide & microcrystalline | | | | |
| | cellulose & cellulose gum | | | | |
| LCW AC-5 Sericite FSE | Mica & microcrystalline | 2.50 | 2.50 | 2.50 | 2.50 |
| | cellulose & cellulose gum | | | | |
| Aerosil R 812 S VV 60 | Silica silylate | 5.00 | 5.00 | 5.00 | 5.00 |
| Abil 20 | Dimethicone | 2.00 | 2.00 | 2.00 | 2.00 |
| Abil B 8843 | Dimethicone copolyol | 0.50 | 0.50 | 0.50 | 0.50 |
| Liquipar oil | “Parabens” | 0.15 | 0.15 | 0.15 | 0.15 |
| LCW Covagel | Sodium carboxymethyl starch | 0.75 | 0.75 | 0.75 | 0.75 |
| Total | | 100.0 | 100.0 | 100.0 | 100.0 |

### Examples 9 and 10

### Blusher/Lip Colour

| | | 9 | 10 |
|---|---|---|---|
| Raw material | INCI name | wt. % | wt. % |
| Demineralised water | Aqua (water) | 82.75 | 78.50 |
| Hydrolite-5 | Pentylene glycol | 5.00 | 5.00 |
| LCW AC-5 Red | CI 77491 & microcrystalline | 3.25 | 3.25 |
| R-516 L | cellulose & cellulose gum | | |
| LCW AC-5 Black | CI 77499 & microcrystalline | 0.10 | 0.10 |
| BL-100 | cellulose & cellulose gum | | |
| LCW AC-5 TiO2 | CI 77891 & aluminium | 2.00 | 2.25 |
| CR-50 | hydroxide & microcrystalline | | |
| | cellulose & cellulose gum | | |
| LCW AC-5 Sericite | Mica & microcrystalline | | 2.50 |
| FSE | cellulose & cellulose gum | | |
| Aerosil R 812 S, | Silica silylate | 5.00 | 5.00 |
| # 2409 | | | |
| Abil 20 | Dimethicone | 0.50 | 2.00 |
| Abil B 8843 | Dimethicone copolyol | 0.50 | 0.50 |
| Liquipar oil | Isobutylparaben & | 0.15 | 0.15 |
| | isopropylparaben & | | |
| | butylparaben | | |
| LCW Covagel | Sodium carboxymethyl starch | 0.75 | 0.75 |
| Total | | 100.00 | 100.00 |

### Examples 11 and 12

### Eye Shadows

| | | 11 | 12 |
|---|---|---|---|
| Raw material | INCI name | wt. % | wt. % |
| Demineralised water | Aqua (water) | 83.10 | 79.10 |
| Hydrolite-5 | Pentylene glycol | 5.00 | 5.00 |
| Colorona Majestic Green | CI 77891, 77019, 77288 | 5.00 | 5.00 |
| LCW AC-5 Sericite FSE | Mica & microcrystalline | | 2.50 |
| | cellulose & cellulose gum | | |
| Aerosil R 812 S, # 2409 | Silica silylate | 5.00 | 5.00 |
| Abil 20 | Dimethicone | 0.50 | 2.00 |
| Abil B 8843 | Dimethicone copolyol | 0.50 | 0.50 |
| Liquipar oil | Isobutylparaben & | 0.15 | 0.15 |
| | isopropylparaben & | | |
| | butylparaben | | |
| LCW Covagel | Sodium carboxymethyl | 0.75 | 0.75 |
| | starch | | |
| Total | | 100.00 | 100.00 |

### Example 13 and 14

### Skin Lightener

| | | 13 | 14 |
|---|---|---|---|
| Raw material | INCI name | wt. % | wt. % |
| Demineralised water | Aqua (water) | 86.50 | 84.00 |
| Hydrolite-5 | Pentylene glycol | 5.00 | 5.00 |
| Cosmocair C 250 | 1-Methylhydantoine-2-imide | 0.10 | 0.10 |
| LCW AC-5 Sericite | Mica & microcrystalline | | 2.50 |
| FSE | cellulose & cellulose gum | | |
| Aerosil R 812 S | Silica silylate | 5.00 | 5.00 |
| VV 60 | | | |
| Abil 20 | Dimethicone | 2.00 | 2.00 |
| Abil B 8843 | Dimethicone copolyol | 0.50 | 0.50 |
| Liquipar oil | Isobutylparaben & | 0.15 | 0.15 |
| | isopropylparaben & | | |
| | butylparaben | | |
| Covagel | Sodium carboxymethyl starch | 0.75 | 0.75 |
| Total | | 100.00 | 100.00 |

### Example 15 and 16

### Face & Body Toner

| | | 15 | 16 |
|---|---|---|---|
| Raw material | INCI name | wt. % | wt. % |
| Demineralised water | Aqua (water) | 76.60 | 74.10 |
| Hydrolite-5 | Pentylene glycol | 5.00 | 5.00 |
| Hamamelis extract | Hamamelis virginiana | 10.00 | 10.00 |
| LCW AC-5 Sericite FSE | Mica & microcrystalline | | 2.50 |
| | cellulose & cellulose gum | | |
| Aerosil R 812 S VV 60 | Silica silylate | 5.00 | 5.00 |
| Abil 20 | Dimethicone | 2.00 | 2.00 |
| Abil B 8843 | Dimethicone copolyol | 0.50 | 0.50 |
| Liquipar oil | Isobutylparaben & | 0.15 | 0.15 |
| | isopropylparaben & | | |
| | butylparaben | | |
| Covagel | Sodium carboxymethyl | 0.75 | 0.75 |
| | starch | | |
| Total | | 100.0 | 100.0 |

### Example 17-20

### Sunscreen Formulation Containing Micropigments

| | | 17 | 18 | 19 | 20 |
|---|---|---|---|---|---|
| Raw material | INCI name | wt. % | wt. % | wt. % | wt. % |
| Demineralised water | Aqua (water) | 78.60 | 76.10 | 73.60 | 71.10 |
| Hydrolite-5 | Pentylene glycol | 5.00 | 5.00 | 5.00 | 5.00 |
| Phenylbenzimidazole | TEA phenylbenzimidazole | 8.00 | 8.00 | 8.00 | 8.00 |
| sulfonic acid | sulfonate | | | | |
| triethanolamine salt | | | | | |
| (Merck KGaA) | | | | | |
| Tego Sun TAQ 40 | Titanium dioxide & glycerine | | | 5.00 | 5.00 |
| | & isolaureth-4 phosphate & | | | | |
| | vinyl buteth-25/sodium | | | | |
| | maleate copolymer | | | | |
| LCW AC-5 Sericite FSE | Mica & microcrystalline | | 2.50 | | 2.50 |
| | cellulose & cellulose gum | | | | |
| Aerosil R 812 S VV 60 | Silica silylate | 5.00 | 5.00 | 5.00 | 5.00 |
| Abil 20 | Dimethicone | 2.00 | 2.00 | 2.00 | 2.00 |
| Abil B 8843 (DC 193) | Dimethicone copolyol | 0.50 | 0.50 | 0.50 | 0.50 |
| Liquipar oil | Isobutylparaben & | 0.15 | 0.15 | 0.15 | 0.15 |
| | isopropylparaben & | | | | |
| | butylparaben | | | | |
| Covagel | Sodium carboxymethyl starch | 0.75 | 0.75 | 0.75 | 0.75 |
| Total | | 100.0 | 100.0 | 100.0 | 100.0 |

### Example 21-24

### Sunscreen Formulations Containing Micropigments

| | | 21 | 22 | 23 | 24 |
|---|---|---|---|---|---|
| Raw material | INCI name | wt. % | wt. % | wt. % | wt. % |
| Demineralised water | Aqua (water) | 76.60 | 74.10 | 71.60 | 69.10 |
| Hydrolite-5 | Pentylene glycol | 5.00 | 5.00 | 5.00 | 5.00 |
| Solarplexx | Aqua (water) & ethylhexyl | 10.00 | 10.00 | 10.00 | 10.00 |
| | methoxycinnamate & phenyl | | | | |
| | trimethicone & | | | | |
| | phenylbenzimadazole sulfonic | | | | |
| | acid & triethanolamine & | | | | |
| | phospholipids | | | | |
| Tego Sun TAQ 40 | Titanium dioxide & glycerine | | | 5.00 | 5.00 |
| | & isolaureth-4 phosphate & | | | | |
| | vinyl buteth-25/sodium | | | | |
| | maleate copolymer | | | | |
| LCW Ac-5 Sericite FSE | Mica & microcrystalline | | 2.50 | | 2.50 |
| | cellulose & cellulose gum | | | | |
| Aerosil R 812 S VV 60 | Silica silylate | 5.00 | 5.00 | 5.00 | 5.00 |
| Abil 20 | Dimethicone | 2.00 | 2.00 | 2.00 | 2.00 |
| Abil B 8843 (DC 193) | Dimethicone copolyol | 0.50 | 0.50 | 0.50 | 0.50 |
| Liquipar oil | Isobutylparaben & | 0.15 | 0.15 | 0.15 | 0.15 |
| | isopropylparaben & | | | | |
| | butylparaben | | | | |
| Covagel | Sodium carboxymethyl starch | 0.75 | 0.75 | 0.75 | 0.75 |
| Total | | 100.0 | 100.0 | 100.0 | 100.0 |

### Examples 25 and 26

### Aftersun Preparations Containing Panthenol

| | | 25 | 26 |
|---|---|---|---|
| Raw material | INCI name | wt. % | wt. % |
| Demineralised water | Aqua (water) | 81.60 | 79.10 |
| Hydrolite-5 | Pentylene glycol | 5.00 | 5.00 |
| Panthenol | Panthenol | 5.00 | 5.00 |
| LCW AC-5 Sericite FSE | Mica & microcrystalline | | 2.50 |
| | cellulose & cellulose gum | | |
| Aerosil R 812 S VV 60 | Silica silylate | 5.00 | 5.00 |
| Abil 20 | Dimethicone | 2.00 | 2.00 |
| Abil B 8843 (DC 193) | Dimethicone copolyol | 0.50 | 0.50 |
| Liquipar oil | Isobutylparaben & | 0.15 | 0.15 |
| | isopropylparaben & | | |
| | butylparaben | | |
| Covagel | Sodium carboxymethyl | 0.75 | 0.75 |
| | starch | | |
| Total | | 100.00 | 100.00 |

### Examples 27-30

### Deodorant/Antiperspirant

| | | 27 | 28 | 29 | 30 |
|---|---|---|---|---|---|
| Raw material | INCI name | wt. % | wt. % | wt. % | wt. % |
| Demineralised water | Aqua (water) | 84.60 | 82.10 | 64.60 | 62.10 |
| Hydrolite-5 | Pentylene glycol | 5.00 | 5.00 | 5.00 | 5.00 |
| Tego Deo LYS | Zinc ricinoleate & lysine & | 2.00 | 2.00 | 2.00 | 2.00 |
| | propylene glycol | | | | |
| Locron liquid | Aluminium chlorohydrate & | | | 20.00 | 20.00 |
| | aqua (water) | | | | |
| LCW AC-5 Sericite FSE | Mica & microcrystalline | | 2.50 | | 2.50 |
| | cellulose & cellulose gum | | | | |
| Aerosil R 812 S VV 60 | Silica silylate | 5.00 | 5.00 | 5.00 | 5.00 |
| Abil 20 | Dimethicone | 2.00 | 2.00 | 2.00 | 2.00 |
| Abil B 8843 (DC 193) | Dimethicone copolyol | 0.50 | 0.50 | 0.50 | 0.50 |
| Liquipar oil | Isobutylparaben & | 0.15 | 0.15 | 0.15 | 0.15 |
| | isopropylparaben & | | | | |
| | butylparaben | | | | |
| Covagel | Sodium carboxymethyl starch | 0.75 | 0.75 | 0.75 | 0.75 |
| Total | | 100.0 | 100.0 | 100.0 | 100.0 |

### Examples 31 and 32

### Face & Body Glitter

| | | 31 | 32 |
|---|---|---|---|
| Raw material | INCI name | wt. % | wt. % |
| Demineralised water | Aqua (water) | 76.60 | 74.10 |
| Hydrolite-5 | Pentylene glycol | 5.00 | 5.00 |
| Glitter pigments | Mica & iron oxides | 10.00 | 10.00 |
| LCW AC-5 Sericite FSE | Mica & microcrystalline | | 2.50 |
| | cellulose & cellulose gum | | |
| Aerosil R 812 S VV 60 | Silica silylate | 5.00 | 5.00 |
| Abil 20 | Dimethicone | 2.00 | 2.00 |
| Abil B 8843 (DC 193) | Dimethicone copolyol | 0.50 | 0.50 |
| Liquipar oil | Isobutylparaben & | 0.15 | 0.15 |
| | isopropylparaben & | | |
| | butylparaben | | |
| Covagel | Sodium carboxymethyl | 0.75 | 0.75 |
| | starch | | |
| Total | | 100.00 | 100.00 |

### Examples 33 and 34

### Handcare and Footcare Product Containing Urea

| | | 33 | 34 |
|---|---|---|---|
| Raw material | INCI name | wt. % | wt. % |
| Demineralised water | Aqua (water) | 81.60 | 79.10 |
| Hydrolite-5 | Pentylene glycol | 5.00 | 5.00 |
| Urea | Urea | 5.00 | 5.00 |
| LCW AC-5 Sericite FSE | Mica & microcrystalline | | 2.50 |
| | cellulose & cellulose gum | | |
| Aerosil R 812 S VV 60 | Silica silylate | 5.00 | 5.00 |
| Abil 20 | Dimethicone | 2.00 | 2.00 |
| Abil B 8843 (DC 193) | Dimethicone copolyol | 0.50 | 0.50 |
| Liquipar oil | Isobutylparaben & | 0.15 | 0.15 |
| | isopropylparaben & | | |
| | butylparaben | | |
| Covagel | Sodium carboxymethyl | 0.75 | 0.75 |
| | starch | | |
| Total | | 100.00 | 100.00 |

### Example 35 and 36

### Self-Tanner

| | | 35 | 36 |
|---|---|---|---|
| Raw material | INCI name | wt. % | wt. % |
| Demineralised water | Aqua (water) | 83.60 | 81.10 |
| Hydrolite-5 | Pentylene glycol | 5.00 | 5.00 |
| Dihydroxyacetone | Dihydroxyacetone | 3.00 | 3.00 |
| LCW AC-5 Sericite FSE | Mica & microcrystalline | | 2.50 |
| | cellulose & cellulose gum | | |
| Aerosil R 812 S VV 60 | Silica silylate | 5.00 | 5.00 |
| Abil 20 | Dimethicone | 2.00 | 2.00 |
| Abil B 8843 (DC 193) | Dimethicone copolyol | 0.50 | 0.50 |
| Liquipar oil | Isobutylparaben & | 0.15 | 0.15 |
| | isopropylparaben & | | |
| | butylparaben | | |
| Covagel | Sodium carboxymethyl | 0.75 | 0.75 |
| | starch | | |
| Total | | 100.00 | 100.00 |

### Example 37 and 38

### Skincare Product Containing Fruit Acid

| | | 37 | 38 |
|---|---|---|---|
| Raw material | INCI name | wt. % | wt. % |
| Demineralised water | Aqua (water) | 76.60 | 74.10 |
| Hydrolite-5 | Pentylene glycol | 5.00 | 5.00 |
| Mixed fruit acid | Glycolic acid & lactic acid | 10.00 | 10.00 |
| | & citric acid & aqua | | |
| | (water) | | |
| LCW AC-5 Sericite FSE | Mica & microcrystalline | | 2.50 |
| | cellulose & cellulose gum | | |
| Aerosil R 812 S VV 60 | Silica silylate | 5.00 | 5.00 |
| Abil 20 | Dimethicone | 2.00 | 2.00 |
| Abil B 8843 (DC 193) | Dimethicone copolyol | 0.50 | 0.50 |
| Liquipar oil | Isobutylparaben & | 0.15 | 0.15 |
| | isopropylparaben & | | |
| | butylparaben | | |
| Covagel | Sodium carboxymethyl | 0.75 | 0.75 |
| | starch | | |
| Total | | 100.00 | 100.00 |

### Example 39 and 40

### Tooth Whitener

| | | 39 | 40 |
|---|---|---|---|
| Raw material | INCI name | wt. % | wt. % |
| Demineralised water | Aqua (water) | 76.60 | 74.10 |
| Hydrolite-5 | Pentylene glycol | 5.00 | 5.00 |
| Hydrogen peroxide | Hydrogen peroxide | 10.00 | 10.00 |
| (30 wt. %) | | | |
| LCW AC-5 Sericite FSE | Mica & microcrystalline | | 2.50 |
| | cellulose & cellulose gum | | |
| Aerosil R 812 S VV 60 | Silica silylate | 5.00 | 5.00 |
| Abil 20 | Dimethicone | 2.00 | 2.00 |
| Abil B 8843 (DC 193) | Dimethicone copolyol | 0.50 | 0.50 |
| Liquipar oil | Isobutylparaben & | 0.15 | 0.15 |
| | isopropylparaben & | | |
| | butylparaben | | |
| Covagel | Sodium carboxymethyl | 0.75 | 0.75 |
| | starch | | |
| Total | | 100.00 | 100.00 |

1. A brushable cosmetic preparation, comprising:
50-95 wt. % water,
hydrophobed silicon dioxide powder,
at least one cosmetically relevant active ingredient or auxiliary substance that is water-soluble or dispersible or emulsifiable in an aqueous medium, and
a viscosity regulator;
wherein said preparation has a creamy or pasty consistency.

2. The preparation according to claim 1, wherein the proportion of water is 75 to 90 wt. %, based on the total weight of said preparation.

3. The preparation according to claims 1 or 2, wherein the proportion of hydrophobed silicon dioxide powder is 1 to 15 wt. %, based on the total weight of said preparation.

4. The preparation according to claim 1, wherein the compacted bulk density, determined according to DIN EN ISO 787-11, is at least 70 g/l.

5. The preparation according to claim 1, wherein the proportion of cosmetically relevant active ingredient or auxiliary substance that is water-soluble or is dispersible or emulsifiable in an aqueous medium is 1 to 25 wt. %, based on the total weight of said preparation.

6. The preparation according to claim 1, wherein the proportion of viscosity regulator is 0.1 to 10 wt. %, based on the total weight of said preparation.

7. The preparation according to claim 1, wherein the preparation comprises 0.1 to 10 wt. % of at least one oil body, based on the total weight of said preparation.

8. The preparation according to claim 1, wherein the preparation comprises at least 0.1 to 10 wt. % of a pigments based on the total weight of said preparation.

9. A process, for the production of the preparation according to claim 1, comprising:
adding at least one cosmetically relevant active ingredient and/or auxiliary substance and optionally at least one pigment in water to a solution, dispersion or emulsion;
adding hydrophobed silicon powder in portions or continuously whilst mixing, to obtain a mixture in powder form, and
continuing mixing is continued until the mixture takes on a pasty consistency;
adding under mixing a viscosity regulator and optionally at least one oil body, optionally before, together with or after addition of the viscosity regulator.

10. A paste, cream, gel, pencil, foam, emulsion, dispersion roll-on or lotion, comprising the composition according to claim 1.

11. The preparation according to claim 1, wherein said hydrophobed silicon dioxide powder is a powder that when added to water forms a product that is initially in powder form.

12. The preparation according to claim 1, wherein said hydrophobed silicon dioxide powder is silanized.

13. The preparation according to claim 1, wherein said hydrophobed silicon dioxide powders preferably display a methanol wettability of at least 40.

14. The preparation according to claim 1, wherein said cosmetically relevant active ingredient or auxiliary substance is selected from the group consisting of UV light stabilising filters, dyes, pigments, humectants, skin moisturising agents, deodorizing agents, antiperspirant agents, biogenic substances, insect repellent agents, hydrotropes, anti-dandruff agents, bleaching agents, skin lightening agents, self-tanning agents, preservatives, surfactants, emulsifiers, perfume oils, plant extracts and mixtures thereof.

15. The preparation according to claim 1, further comprising 0.1 to 15 wt. % of a humectant.

16. The preparation according to claim 1, further comprising a member selected from the group consisting of glycerol, diglycerol, triglycerol, butylene glycol and mxtures thereof.

17. The preparation according to claim 1, wherein said oil body is a substance or mixtures of substances that are liquid at 20° C. and are immiscible with water at 25° C.

18. The preparation according to claim 1, comprising a viscosity regulator which is a hydrogel former or a hydrocolloid.

19. The preparation according to claim 1, wherein said cosmetically relevant active ingredient or auxiliary substance is water soluble.

20. The preparation according to claim 1, wherein said cosmetically relevant active ingredient or auxiliary substance is dispersible in an aqueous medium.
